(19) United States
(12) Patent Application Publication
(10) Pub. No.: US 2023/0149464 A1
(43) Pub. Date: May 18, 2023

(54) FEEDER FREE CELL CULTURE METHODS FOR EXPANDING NATURAL KILLER CELL PREPARATIONS

(71) Applicant: Janssen Biotech, Inc., Horsham, PA (US)

(72) Inventors: Andrew W. SNOWDEN, Woodlawn Hills, CA (US); Ali ZAMANI, Lansdowne, PA (US)

(21) Appl. No.: 17/988,910

(22) Filed: Nov. 17, 2022

Related U.S. Application Data
(60) Provisional application No. 63/281,002, filed on Nov. 18, 2021.

(51) Int. Cl. A61K 35/17; C12N 5/0783; A61P 35/00

(52) U.S. Cl. CPC A61K 35/17; C12N 5/0646; A61P 35/00; C12N 2501/998; C12N 2533/20; C12N 2501/599; C12N 2501/2302; C12N 2501/2312; C12N 2501/2315; C12N 2501/2318; C12N 2501/2321

(57) ABSTRACT

The present disclosure is directed to a feeder-cell free methods of producing an expanded natural killer (NK) cell preparation. This method comprises providing a starting preparation of NK cells and treating the starting preparation with a natural killer cell p30-related protein (NKp30) modulating agent alone or with other expansion agents as described herein. The method further involves culturing the treated preparation under conditions effective to expand the starting preparation of NK cells to produce an expanded NK cell preparation. Other aspects of the disclosure related to therapeutic preparations of NK cells produced in accordance with the methods described herein.

### CROSS-REFERENCE TO RELATED APPLICATIONS

This application claims the benefit of U.S. Provisional Application Ser. No. 63/281,002, filed 18 Nov. 2021. The entire contents of the aforementioned applications is incorporated herein by reference in its entirety.

### FIELD

The present disclosure relates to methods for feeder free expansion of natural killer (NK) cells derived from iPSC (iNK) or from cord blood (CB-NK) to produce therapeutic NK cells preparations.

### BACKGROUND

Natural killer (NK) cells are cytotoxic lymphocytes that play a critical role in the innate immune response. NK cells are phenotypically characterized as CD56⁺CD3⁻ and have the capability to spontaneously lyse tumor cells and virally infected cells. NK cells are also involved in adaptive immune response by a process called antibody-dependent cellular cytotoxicity, where NK cells directly bind to and kill cells that are bound by antibody molecules. In the human body, NK cells differentiate and mature in various immune organs, such as bone marrow, lymph node, spleen, tonsils, and thymus.

Human induced pluripotent stem cells (iPSC) are a type of pluripotent stem cells, which are generated artificially from a non-pluripotent cell by forced expression of specific genes. iPSC can be utilized to generate potentially unlimited source of therapeutically viable NK cells for treating various types of cancer as well as acute and chronic viral infections. Thus, it is imperative to be able to efficiently and reproducibility generate NK cells from iPSC, in a controllable manner.

The process of NK cell differentiation initiating from iPSC in a feeder cell-dependent manner has been documented. Existing protocols for ex vivo expansion of NK cells from iPSC/CD34⁺ derived immature NK cells rely on co-culture with an artificially generated feeder cell line such as the K562 tumorigenic cell line. Expansion in the feeder cell systems introduces unknown genetic/safety variation into an expanded NK cell population which can negatively impact the subsequent use of the expanded cell population in adoptive cell immunotherapies. Therefore, there is a need for robust methods of ex vivo expansion of NK cells that do not involve co-culture with K562 cells or other feeder cell lines.

The present disclosure is directed at overcoming current deficiencies in the expansion of natural killer cells.

### SUMMARY

A first aspect of the disclosure is directed to a method of producing an expanded natural killer (NK) cell preparation. This method comprises providing a starting preparation of NK cells and treating the starting preparation with a natural killer cell p30-related protein (NKp30) modulating agent. The method further involves culturing the treated preparation under conditions effective to expand the starting preparation of NK cells to produce an expanded NK cell preparation. In any embodiment, the starting NK cell preparation is a preparation derived from induced pluripotent stem cells or from cord blood. In any embodiment, the NKp30 modulating agent is an anti-NKp30 antibody.

Another aspect of the present disclosure is directed to a therapeutic preparation of NK cells produced according to the methods disclosed here.

Another aspect of the present disclosure is directed to a pharmaceutical composition comprising the expanded preparation of NK cells produced according to the methods disclosed here and a pharmaceutically acceptable carrier.

Another aspect of the present disclosure is directed to a method of treating a subject in need of adoptive NK cell therapy. This method involves administering to the subject in need of adoptive NK cell therapy, the therapeutic preparation of NK cells produced in accordance with the methods disclosed herein or a pharmaceutical composition comprising the same in an amount effective to treat the subject in need of adoptive NK cell therapy.

Disclosed herein are methods for expanding NK cells from a heterogeneous preparation of immature NK cells derived from CD34⁺ progenitor cells derived from iPSC or cord blood. These methods are unique from prior art methods which require co-culture of the NK cells with a feeder cell population, e.g., a genetically modified feeder cell line (K562) or B cell lines naturally immortalized by infection with Epstein-Barr virus. The feeder free culture method described herein produces a heterogeneous expanded population of late stage immature NK cells and mature NK cells that are advantageous for adoptive cell therapy given their persistence and expansion capacity upon in vivo transplantation. In contrast, NK cells expanded in feeder cultures comprise a more homogenous population of mature NK cells that have limited expansion and/or are exhausted quickly following patient treatment. Because the expanded NK cell preparations of the present disclosure are produced in the absence of the tumorigenic feeder cells, there is no risk of feeder cell contamination, and thus, no need to employ a stringent purification process prior to therapeutic transplantation of the expanded NK cell preparation. The expanded NK cell preparations produced in accordance with the feeder-free methods described herein can be utilized as “off-the-shelf” therapeutic NK cell compositions that are suitable for administration at multiple doses to individuals across different genetic backgrounds and immunogenic barriers.

### BRIEF DESCRIPTION OF THE DRAWINGS

FIG. 1 is a graph showing the fold increase in NK cells derived from cord blood CD34+ over 10 days in culture with a first round of treatment comprising the specified combinations of expansion agents. Expansion of NK cells in the presence of feeder cell (i.e., K562 cells) was carried out in parallel for comparison. NKp30=Anti NKp30 biotinylated antibody conjugated to anti biotin beads; 2B4=Anti 2B4 (CD244) biotinylated antibody conjugated to anti biotin beads; CD48=CD48-Fc protein conjugated to anti Fc beads; CD155=CD155-Fc protein conjugated to anti Fc beads; ICAM2=ICAM-2-Fc protein conjugated to anti Fc beads; 4-1BBL=4-1BBL-Fc protein conjugated to anti Fc beads; K562-4-1BBL-IL21 or K562=feeder cell cultures.

FIG. 2 is a graph showing the fold increase in NK cells (cultured in the presence of NKp30/2B4 in first round as described in reference to FIG. 1) over an additional 10 days of culture after a second round of treatment with the specified combination of expansion agents. NKp30=Anti NKp30 biotinylated antibody conjugated to anti biotin beads; CD2=Anti CD2 biotinylated antibody conjugated to anti biotin beads; CD48=CD48-Fc protein conjugated to anti Fc beads; NKp46=Anti NKp46 biotinylated antibody conjugated to anti biotin beads; NKp44=Anti NKp44 biotinylated antibody conjugated to anti biotin beads; NKp80=Anti NKp80 biotinylated antibody conjugated to anti biotin beads.

FIG. 3 is a graph showing the fold increase in NK cells derived from iPSC (iNK) at days 3 and 10 of culture in the presence of CD48, CD155, and NKp30 each conjugated to either LigaTrap beads (Dianova GmbH, Hamburg, Germany), Protein A/G beads, or Anti human Fc-antibody.

FIG. 4A is a graph showing the fold increase in NK cells derived from iPSC (iNK) at days 10 of culture in the presence of NKp30 for initial three days following by addition of anti DNAM1 biotinylated antibody conjugated to anti biotin beads (DNAM1) or CD48 or 4-1BBL each conjugated to LigaTrap beads (Dianova GmbH, Hamburg, Germany).

FIG. 4 B is a graph showing the fold increase in NK cells derived from iPSC (iNK) at days 10 of culture in the presence of NKp30 for initial three days following by addition of IL21 or 4-1BBL or combination of IL21 and 4-1BBL or CD40 or CRACC each conjugated to LigaTrap beads (Dianova GmbH, Hamburg, Germany).

FIGS. 5A-5D show a flow cytometry analysis of NK surface markers (FIGS. 5A-5C) and exhaustion markers (FIG. 5D), sub gated on CD56+CD3− population of NK cells produced from iPSC using the feeder-free method described herein. The analysis was carried at day 10 of culture, where the starting population iNK had been cultured in the presence of NKp30 for the initial three days followed by addition of anti DNAM1 biotinylated antibody conjugated to anti biotin beads. FIG. 5A shows flow cytometry analysis of the expression levels of general NK markers sub gated on CD56 positive cells (CD45, NKP30, DNAM1, 2B4). FIG. 5B shows expression levels of immature NK markers (NKG2A, NKG2D, NKP46, NKP44) and FIG. 5C are graphs depicting expression of mature NK markers (NKp80, CD16, KIR2DL2, CRACC). FIG. 5D is a graph showing expression levels of exhaustion markers on expanded NK. This marker analysis shows that the expanded NK population contains a combination of mature and immature phenotypes with no significant exhausted population, pointing to a further expansion potential.

FIG. 6 is a graph showing cytotoxicity effect of expanded NK cells derived from iPSC (iNK). The starting NK preparation was cultured in the presence of NKp30 for an initial three days followed by the addition of anti-DNAM1 biotinylated antibody conjugated to anti-biotin beads for an additional seven days of culture. In this killing assay, the expanded NK preparation was co-cultured with K562 cancer cell line (“target” cells) at a different ratios (NK:K562 of 0.25:1 to 10:1) to determine the cytotoxic effect of the expanded NK preparation on the target K562 cells. At each time point (every 6 hours), the levels of live cancer cells were determined using real-time live imaging system (Incucyte, Sartorius, US) and normalized to the initial loaded number of target cells at time zero (at seeding day).

### DETAILED DESCRIPTION

The present disclosure relates to methods of expanding natural killer cells derived from iPSCs or cord blood in a feeder free cell culture environment. Natural killer (hereinafter abbreviated as “NK”) cells are lymphoid cells that participate in immune reactions. These cells have variety of functions, including the killing of tumor cells, cells undergoing oncogenic transformation, virally infected cells, and other abnormal cells in a living body. Thus NK cells are important components of innate immunological surveillance mechanisms. NK cells exhibit spontaneous non-MHC-restricted cytotoxic activity against virally infected and tumor cells, and mediate resistance to viral infections and cancer development in vivo. Thus, ex vivo methods for effectively expanding or increasing the number of NK cells is useful to generate a therapeutically effective concentrations of cells suitable for immunotherapeutic treatment of tumors and viral infection.

Accordingly, a first aspect of the disclosure is directed to a method of producing an expanded natural killer (NK) cell preparation. This method comprises providing a starting preparation of NK cells and treating the starting preparation with a natural killer cell p30-related protein (NKp30) modulating agent. The method further involves culturing the treated preparation under conditions effective to expand the starting preparation of NK cells to produce an expanded NK cell preparation.

In accordance with this and all aspects of the present disclosure the method of producing the expanded NK cell preparation does not involve culturing the starting preparation of NK cells in the presence of a feeder cell population of cells at any time during the method. The method is carried out in the complete absence of a feeder cell population.

In some embodiments, the starting preparation of NK cells is a preparation of immature NK (iNK) cells. Immature NK cells include NK progenitor cells of Stages 3 and 4, as defined by Abel et al., “Natural Killer Cells: Development, Maturation, and Clinical Utilization,” Frontiers Immunol. 9:1869 (2018), which is hereby incorporated by reference in its entirety, that are capable of giving rise to mature NK cells. Immature NK cells are defined by their expression of any one or more of NKG2D, CD335, CD337, NKG2A, NKP80 and CD56^{bright} In some embodiments, the starting preparation of NK cells is a preparation of mature NK cells. Mature NK cells are committed NK cells, having characteristic surface markers and NK cell function, and lacking the potential for further differentiation (Abel et al., “Natural Killer Cells: Development, Maturation, and Clinical Utilization,” Frontiers Immunol. 9:1869 (2018), which is hereby incorporated by reference in its entirety). Mature NK cells are defined by their expression of any one or more of CD16, CD56^{dim}, KIR, and CD57. In some embodiments, the starting preparation of NK cells is a preparation containing a mixture of immature and mature NK cells. In some embodiments, the starting preparation of NK cells is characterized by a CD56^{+/-}/CD16⁻/CD45⁺/CD34⁻ expression profile. In some embodiments, the starting preparation of NK cells is characterized by a CD56⁺/CD16⁻/CD45⁺/CD34⁻ expression profile.

In any embodiment, the NK cells of the starting NK cell preparation are mammalian NK cells derived from iPSC or CD34+ hematopoietic progenitor cells of cord blood. Suitable mammalian NK cells populations include, without limitation human NK cells, primate NK cells, bovine NK cells, canine NK cells, feline NK cells, rodent NK cells (e.g., murine NK cells), as well as NK cells derived from other mammals. In a preferred embodiment, the NK cells of the starting NK preparation are human NK cells and the NK cells of the expanded NK preparation are likewise human NK cells.

In any embodiment, the starting preparation of NK cells is derived from a CD34⁺ hematopoietic progenitor cell population. In any embodiment, the CD34⁺ hematopoietic progenitor cell population is a population of CD34⁺ cord blood cells. Methods of producing NK cells from CD34⁺ hematopoietic progenitor cells are well known in the art, see e.g., Spanholtz et al., “Clinical-grade generation of active NK cells from cord blood hematopoietic progenitor cells for immunotherapy using a closed-system culture process,” PLoS One 6(6):e20740 (2011); Cany et al., “Combined IL-15 and IL-12 drives the generation of CD34-derived natural killer cells with superior maturation and alloreactivity potential following adoptive transfer,” Oncoimmunology 4(7):e1017701 (2015); and Spanholtz et al., “High log-scale expansion of functional human natural killer cells from umbilical cord blood CD34-positive cells for adoptive cancer immunotherapy,” PLoS One 5(2):e9221 (2010), which are hereby incorporated by reference in their entirety.

In any embodiment, the CD34⁺ hematopoietic progenitor cell population is derived from or differentiated from a population of induced pluripotent stem cell (iPSCs). Methods of generating natural killer cells from iPSCs populations is well known in the art (see e.g., Rezvani et al., “Engineering natural killer cells for cancer immunotherapy,” Mol Ther. 25(8):1769-81 (2017); Li et al. “Human iPSC-derived natural killer cells engineered with chimeric antigen receptors enhance anti-tumor activity,” Cell Stem Cell, 23(2):181-192.e5 (2018); Ni et al. “Expression of chimeric receptor CD4zeta by natural killer cells derived from human pluripotent stem cells improves in vitro activity but does not enhance suppression of HIV infection in vivo,” Stem Cells 32(4):1021-31 (2014), which are hereby incorporated by reference in their entirety).

In any embodiment, the NK cells of the starting NK cell preparation are genetically modified, i.e., containing and/or expressing a foreign gene or nucleic acid sequence which in turn modifies the genotype or phenotype of the cell or its progeny. For example, in one embodiment, the NK cells of the starting NK cell preparation are genetically modified to express a chimeric antigen receptor (CAR). In another embodiment, the NK cells of the starting NK cell preparation are genetically modified to express or overexpress one or more chemokine receptors. Methods of producing genetically modified NK cells are well known in the art and suitable for use in the methods described herein (see e.g., Rezvani et al., “Engineering natural killer cells for cancer immunotherapy,” Mol Ther. 25(8):1769-81 (2017); Li et al. “Human iPSC-derived natural killer cells engineered with chimeric antigen receptors enhance anti-tumor activity,” Cell Stem Cell, 23(2):181-192.e5 (2018); Ni et al. “Expression of chimeric receptor CD4zeta by natural killer cells derived from human pluripotent stem cells improves in vitro activity but does not enhance suppression of HIV infection in vivo,” Stem Cells 32(4):1021-31 (2014); Schonfeld et al., “Selective inhibition of tumor growth by clonal NK cells expressing an ErbB2/HER2-specific chimeric antigen receptor,” Mol Ther. 23(2):330-8 (2015); Romanski et al., “CD19-CAR engineered NK-92 cells are sufficient to overcome NK cell resistance in B-cell malignancies,” J Cell Mol Med. 20(7):1287-94 (2016), which are hereby incorporated by reference in their entirety). In accordance with the methods described herein, cells of the expanded NK preparation retain the genetic modification of the starting preparation cells. Thus, in some embodiments, the methods of the present disclosure are suitable in expanding genetically modified NK cells, such as CAR-NK cells, to produce an expanded preparation of genetically modified NK cells.

In any embodiment the starting preparation of NK cells comprising a population of purified NK cells. NK cells can be purified from a sample comprising NK cells and other cells, such as e.g., PMBC or whole blood, by magnetic cell separation methods such as MACS® (Miltenyi Biotec) or flow cytometry methods such as FACS™ These and other methods for purification of cells, e.g. NK cells, are well known in the art.

In any embodiment the concentration of said NK cells in the starting preparation is anywhere from about 1×10⁵ to about 1×10⁶ cells in 5-20 ml of a standard NK cell culture medium. In any embodiment, the concentration of said NK cells in the starting preparation is about 2.5×10⁵ to about 7.5×10⁵ cells in 5-20 ml of a standard NK cell culture medium. In any embodiment, the concentration of said NK cells in the starting preparation is about 1×10⁵, 1.5×10⁵, 2×10⁵, 2.5×10⁵, 3×10⁵, 3.5×10⁵, 4×10⁵, 4.5×10⁵, 5×10⁵, 5.5×10⁵, 6×10⁵, 6.5×10⁵, 7×10⁵, 7.5×10⁵, 8×10⁵, 8.5×10⁵, 9×10⁵, 9.5×10⁵, or 1×10⁶ cells in 5-20 ml. In any embodiment, the concentration of said NK cells in the starting preparation is 5×10⁵ cells in 5-20 ml of a standard NK cell culture medium.

The term “cell culture medium” as used herein includes liquids providing the chemical conditions which are required for NK cell maintenance. Examples of chemical conditions known to support NK cell expansion include but are not limited to solutions, buffers, serum, serum components, nutrients, vitamins, cytokines and other growth factors which are regularly provided in (or may be given manually to) the cell culture medium. Cell culture media suitable for use in the method of expanding NK cells as described herein includes, without limitation, NK-MACS (Miltenyi), TexMACS (Miltenyi), CellGro SCGM (CellGenix), X-Vivo 10, X-Vivo 15, BINKIT NK Cell Initial Medium (Cosmo Bio USA), AIM-V (Invitrogen), DMEM/F12, NK Cell Culture Medium (Upcyte Technologies).

As described supra, the method of expanding the NK cells in accordance with the methods disclosed herein involves treating the starting preparation with an NKp30 modulating agent. NKp30 (also known as Natural cytotoxicity triggering receptor 3, Activating natural killer receptor 30, and CD337), is a cell membrane receptor of NK cells that is activated by binding its extracellular ligands. The extracellular ligands of NKp30 include Large proline-rich protein BAG6 (“BAG6”) and Natural cytotoxicity triggering receptor 3 ligand 1 (also known B7 homolog 6 or B7-H6). As described herein, a suitable NKp30 modulating agent is an anti-NKp30 antibody, anti-NKp30 antibody fragment (e.g., a NKp30 Fab fragment, NKp30 single variable domain, etc.) or anti-NKp30 antibody-based molecule (e.g., NKp30 single-chain antibody). Suitable anti-NKp30 antibodies, antibody fragments and antibody-based molecules are known in the art and commercially available (see e.g., Miltenyi Biotec, Abcam, Bio-Techne (R&D), and Biolegend) for use in the methods disclosed herein. In any embodiment, the NKp30 modulating agent suitable for use in accordance with the methods disclosed herein is a ligand of NKp30, e.g., BAG6 or B7-H6, or NKp30 receptor binding fragments of BAG6 or B7-H6. Suitable recombinant BAG6 and B7-H6 proteins, in particular recombinant human BAG6 and B7-H6 proteins, suitable for use in the in vitro cell culture methods describe herein are known in the art and commercially available from, e.g., R&D Systems and Abcam.

In some embodiments, the starting preparation is treated with the NKp30 modulating agent. In some embodiments, the starting preparation is treated with NKp30 modulating agent in conjunction with a second or third expansion agent. In some embodiments, the NKp30 modulating agent is administered alone to the starting preparation for a one or more rounds of expansion (i.e., 10 days), and then in subsequent rounds of expansion, the NKp30 modulating agent is administered in conjunction with a second or third expansion agent. In yet another embodiment, the NKp30 modulating agent is administered to the starting NK preparation for a one or more rounds of expansion (i.e., 10 days), and then in subsequent rounds of expansion, the NKp30 modulating agent is removed and/or replace by a second and/or third expansion agent.

Suitable second and third expansion agents for use in accordance with the method disclosed herein include, without limitation, a NK cell receptor 2B4 (“2B4”) ligand and a DNAM-1 ligand.

NK cell receptor 2B4, which is also known as SLAM family member 4 and signaling lymphocytic activation molecule 4, is a heterophilic receptor involved in activating NK cells and stimulating NK cell cytotoxicity. CD48 is the ligand of 2B4, and thus, in any embodiment, a suitable 2B4 ligand for use as an expansion agent in the methods disclosed herein is a CD48 protein or 2B4 receptor binding fragment thereof. Recombinant CD48 proteins, in particular recombinant human CD48 proteins, suitable for in vitro cell culture use are known in the art and commercially available from, e.g., R&D Systems and Abcam. In some embodiments, the 2B4 ligand is an anti-2B4 antibody, antibody fragment, or antibody-based molecule. Suitable anti-2B4 antibodies and antibody-based molecules are known in the art and commercially available (see e.g., R&D Systems and Abcam) for purchase and use in the described method.

DNAM-1, which is also known as CD226 antigen, is a cell surface receptor involved in intercellular adhesion, lymphocyte signaling, cytotoxicity and lymphokine secretion. The functional ligands for DNAM-1 include CD155 (also known as Nectin-like protein 5 and Poliovirus receptor) and CD112 (also known as Nectin-2). Accordingly, suitable DNAM-1 ligands for use as expansion agents in the methods disclosed herein include recombinant CD155 and CD112 proteins or DNAM-1 binding fragments of these proteins. Recombinant CD155 and CD112 proteins, in particular recombinant human CD155 and CD112 proteins, suitable for in vitro cell culture use are known in the art and commercially available from, e.g., R&D Systems and Abcam. Other suitable DNAM-1 ligands include DNAM-1 antibodies, antibody fragments, and antibody-based molecules. Suitable anti-DNAM-1 antibodies and antibody-based molecules are known in the art and commercially available for purchase and use in the described method (see e.g., Thermofisher, Abcam, and Biolegend).

In accordance with this and all aspects of the disclosure, it is understood that the use of recombinant protein ligands, e.g., CD155 or CD48, includes the use of fragments, mutants, or variants (e.g., modified forms) of these ligand proteins that retain the ability to modulate their respective NK cell receptor to induce proliferation of NK cells. In other words, suitable ligand fragments, mutants, variants, etc. are those that retain their biological activity as it relates to modulating their respective NK cell receptor to enhance NK cell proliferation in the ex vivo culture conditions.

In any embodiments of the methods described herein, the NKp30 modulating agent is administered to the starting NK preparation in combination with a suitable 2B4 ligand. In any embodiment, the NKp30 modulating agent is administered in conjunction with a DNAM-1 ligand. In any embodiment, the NKp30 modulating agent is administered in conjunction with a 2B4 ligand and a DNAM-1 ligand. In a preferred embodiment, the NKp30 modulating agent is an NKp30 antibody and said antibody is administered to the starting NK cell preparation in conjunction with a recombinant CD48 protein and recombinant CD155 protein.

Other agents that can be used in combination and/or as alternatives to the NKp30 modulating agent, 2B4 ligand, and DNAM-1 ligand in the NK cell expansion method described herein include, without limitation, immobilized IL21, CD40 protein, (binds to CD40 ligand), CRACC/SLAMF7 protein (binds to CRACC/SLAMF7), ligand to the tumor necrosis factor receptor superfamily member 9 (TNFRSF9) and ligands to the tumor necrosis factor receptor superfamily member 4 (TNFRSF4). IL21 is the cytokine that binds to IL21 receptor and common cytokine receptor gamma chain, CD40 is a costimulatory protein that binds to CD40 ligand and CRACC protein is a type transmembrane protein belonging to the CD2 subset of the Ig superfamily. IL21, CD40 and CRACC suitable for in vitro cell culture use are known in the art and commercially available from, e.g., R&D Systems, Acrobiosystems and Sinobiological. The functional ligand for TNFRSF9 is 4-1BB ligand (4-1BBL) or anti-4-1BB antibody and the functional ligand for TNFRSF4 is OX40L. Recombinant 4-1BBL and OX40L proteins, in particular recombinant human 4-1BBL and OX40L proteins or receptor binding fragments as trimer construct (active format) or specific 4-1BB activating antibody (anti-4-1BB antibody) thereof, suitable for in vitro cell culture use are known in the art and commercially available from, e.g., R&D Systems, Biolegend, Acrobiosystems and Abcam. Another expansion agent that can be administered in combination with and/or as an alternative to the NKp30 modulating agent, 2B4 ligand, and DNAM-1 ligand is Intracellular adhesion molecule 2 (ICAM-2), which is the ligand for the leukocyte adhesion protein LFA. Recombinant ICAM-2 proteins, in particular recombinant human ICAM-2 proteins or receptor binding fragments thereof, suitable for in vitro cell culture use are also known in the art and commercially available from, e.g., R&D Systems, Abcam, Acrobiosystems, and Sinobiological.

In any embodiment, the agents disclosed herein that induce NK cell expansion, i.e., NKp30 modulating agent, the 2B4 ligand, DNAM-1 ligand, ICAM-2, 4-1BB ligand, modulating agent (antibody)cra, and OX40L, are collectively referred to herein as “NK cell culture expansion agents”. In any embodiment, these NK cell culture expansion agents as described herein are administered to the starting NK cell preparation in a native, soluble form. In some embodiments, these NK cell culture expansion agents are coupled to a solid support or substrate and administered to the starting NK cell preparation in this substrate bound form. In yet another embodiment, one or more of the NK cell culture expansion agents are administered in a soluble form while one or more are administered to the starting NK cell preparation bound to a solid support.

In accordance with this embodiment of the method, the expansion agents can be coupled to a suitable solid support, e.g., cell culture beads or particles, prior to being administered to the starting NK populations to induce differentiation and expansion of the starting NK cell population. In any embodiment, the NK expansion agents are each coupled or conjugated to their own solid support, e.g., one type of expansion agent is coupled to one type of cell culture bead. In another embodiment, two or more of the NK cell expansion agents are coupled together to one bead type. Coupling of the NK expansion agents to the cell culture beads is routine in the art and can be achieved, for example, via the use of standard binding pair moieties, were a first member of the binding pair moiety is conjugated to the expansion agent (e.g., biotin or an Fc fragment) and the second member of the binding pair moiety (e.g., streptavidin or Fc receptor) is conjugated to the solid support. Suitable binding pair moieties include, without limitation, Fc-IgG Fc receptor, biotin-streptavidin, IgG-Protein A, maltose-maltose binding protein (MBP), albumin-albumin-binding protein (ABP), and calmodulin-calmodulin binding peptide (CBP). Thus, in some embodiments, the expansion agents are coupled to a binding moiety, for example, an Fc portion, a biotin portion, or any other binding pair moiety, where the beads or other solid support contains the partner binding pair moiety for conjugating or coupling the NK expansion agent to the solid support.

Embodiments of the feeder free methods of expanding an NK cell preparation as described herein can further involve treating the starting NK preparation with one or more stimulatory cytokines to supplement the expansion process. According to this embodiment, one or more stimulatory cytokine can be administered to the starting NK cell preparation via addition to the cell culture media at the same time the expansion agents are administered to the cell preparation or anytime during the culturing step (i.e., during any time between day 0 and day 10 of culture). Individual or combinations of cytokines can be added once during the expansion period or repeatedly as needed to maximize expansion. Suitable stimulatory cytokines that can supplement the expansion agents in the method described herein include, without limitation, IL-2, IL-12, IL-15, IL18, IL21, or any combination of these cytokines.

In accordance with the methods of expanding the starting NK cell preparation as described herein, the starting NK cell preparation is treated with one or more of the NK cell culture expansion agents at day zero of the method. The NK cell culture expansion agents are each administered at a concentration, in the cell growth media, suitable for inducing the expansion of the starting NK cell preparation. Typically, a suitable concentration of an individual NK cell culture expansion agent or stimulatory cytokine, e.g., the NKp30 modulating agent, 2B4 ligand, and DNAM-1 ligand, is in the range of between about 0.1 and 1000 ng/mL. In some embodiments, the concentration of each expansion agent is in the range of between about 1 and 200 ng/mL. In some embodiments, the concentration of each expansion agent is in the range of between about 10 and 100 ng/mL, for example, 10 ng/ml, 20 ng/ml, 30 ng/ml, 40 ng/ml, 50 ng/ml, 60 ng/ml, 70 ng/ml, 80 ng/ml, 90 ng/ml, 100 ng/ml, 120 ng/ml, 130 ng/ml, 140 ng/ml, 150 ng/ml, 160 ng/ml, 170 ng/ml, 180 ng/ml, 190 ng/ml, 200 ng/ml, or >200 ng/ml. The Examples below demonstrate exemplary effective concentrations of the expansion agents.

Once the starting population of NK cell is treated with the one or more expansion agents, the treated preparation is cultured under conditions to increase or expand the number of NK cells in the preparation. Conditions effective to increase or expand the number of NK cells in a preparation include standard cell culture conditions, e.g., 37° C., 5% CO₂, and 80% humidity, in the presence of standard cell growth media (e.g., NK MACS medium containing 5% fetal bovine serum).

Culturing of the starting NK cell preparation in the presence of the one or more expansion agents as described herein can be carried out in one or more rounds, where each round is about 5 to about 15 days, e.g., 5 days, 6 days, 7 days, 8 days, 9 days, 10 days, 11 days, 12 days, 13 days, 14 days, 15 days, or >15 days. At the end each round, the expanded NK cell preparation resulting from said culturing step can be collected and split prior to being subjected to one or more additional rounds of expansion. As noted above additional rounds of expansion can be carried out by treating the cells with the same or different expansion reagents. During each round of expansion, the one or expansion reagents are typically only administered to the starting cell preparation once. However, repeated administration of the one or more expansion reagents and/or stimulatory cytokines within one round of expansion is also contemplated.

Culturing the starting preparation of NK cells in the presence of the one or more expansion agents described herein is capable of producing an expanded NK cell preparation comprising at least 5-fold, at least 10-fold, at least 15-fold, at least 20-fold, at least 25-fold, at least 30-fold, at least 35-fold, at least 40-fold, at least 45-fold, at least 50-fold, at least 55-fold, at least 60-fold, at least 65-fold, at least 70-fold, at least 75-fold, at least 80-fold, at least 85-fold, at least 90-fold, at least 95-fold, at least 100-fold or more cells than the number of cells in the starting preparation.

In any embodiment, the method of treating and culturing the starting NK cell preparation is effective to produce an expanded NK cell preparation comprising at least 40-fold more cells than the number of cells in the starting preparation.

In any embodiment, the method of treating and culturing the starting NK cell preparation is effective to produce an expanded NK cell preparation comprising greater than 40-fold more cells than the number of cells in the starting preparation.

In any embodiment, the feeder free method of expanding NK cells described herein produces an expanded NK preparation comprising about 2×10⁹ to about 1×10¹¹ of NK cells. In any embodiment, the method described herein produces an expanded NK preparation comprising 2×10⁹NK cells, 3×10⁹NK cells, 4×10⁹NK cells, 5×10⁹NK cells, 6×10⁹NK cells, 7×10⁹NK cells, 8×10⁹NK cells, 9×10⁹NK cells, 1×10¹⁰ NK cells, 2×10¹⁰ NK cells, 3×10¹⁰ NK cells, 4×10¹⁰ NK cells, 5×10¹⁰ NK cells, 6×10¹⁰ NK cells, 7×10¹⁰ NK cells, 8×10¹⁰ NK cells, 9×10¹⁰ NK cells, 1×10¹¹ NK cells or more than 1×10¹¹NK cells.

In accordance with the methods described herein, the expanded NK cell preparation is a preparation comprising a heterogeneous mixture of immature NK cells and mature NK cells. In one embodiment, the expanded NK cell preparation is a preparation of late stage immature NK cells and mature NK cells. In one embodiment, the expanded NK cell preparation is a preparation of cells characterized by a CD56⁺/CD3⁻/CD45⁺/CD16^{+/-} expression profile. In another embodiment, the NK cells of the expanded NK cell preparation express NKG2-C type II integral membrane protein but do not express NKG2-A/NKG2-B type II integral membrane protein. This expression pattern can be utilized to distinguish the expanded NK cell preparation produced via the methods describe here to an expanded NK cell preparation produced in the presence of a feeder cell population (i.e., NK cells produced in a feeder cell culture system do not express NKG2-C type II integral membrane protein but do express NKG2-A/NKG2-B type II integral membrane protein.

The NK cells of the expanded NK cell preparation generated in accordance with the methods described herein are functional NK cells. In other words, the NK cells of the expanded cell preparation retain their normal biological functions ascribed to NK cells. A non-limiting list of NK cell functions includes, for example, cytotoxicity, induction of apoptosis, cell motility, directed migration, cytokine and other cell signal response, cytokine/chemokine production and secretion, expression of activating and inhibitory cell surface molecules, cell homing and engraftment (in-vivo retention) in a transplanted host, and alteration of disease or disease processes in vivo.

Another aspect of the present disclosure is directed to a therapeutic preparation of NK cells produced according to the methods disclosed here. As referred to herein, a “therapeutic preparation” of NK cells is a preparation comprising a therapeutically effective concentration of functional NK cells. The amount of NK cells in a therapeutic preparation will vary depending on the in vivo use or immunotherapy contemplated. The therapeutic amount administered will also vary depending on the condition of the patient and concurrent therapies and should be determined by considering all appropriate factors.

Another aspect of the present disclosure is directed to a pharmaceutical composition comprising the therapeutic preparation of NK cells and a pharmaceutically acceptable carrier. As used herein, “pharmaceutically acceptable carrier” includes any and all aqueous solvents (e.g., water, saline solutions, parenteral vehicles, such as sodium chloride, Ringer's dextrose, etc.), non-aqueous solvents (e.g., propylene glycol, polyethylene glycol), dispersion media, coatings, surfactants, antioxidants, preservatives (e.g., antibacterial or antifungal agents, anti-oxidants, chelating agents, and inert gases), isotonic agents, absorption delaying agents, and combinations thereof, as would be known to one of ordinary skill in the art. The pH and exact concentration of the various components in a pharmaceutical composition are adjusted according to well-known parameters.

Another aspect of the present disclosure is directed to a method of treating a subject in need of adoptive NK cell therapy. This method involves administering, to the subject in need of adoptive NK cell therapy, the therapeutic preparation of NK cells produced in accordance with the methods disclosed herein or a pharmaceutical composition comprising the same in an amount effective to treat the subject in need of adoptive NK cell therapy.

In one embodiment, the subject in need of adoptive NK cell therapy is a subject having cancer. Infusions of NK cells are a treatment option for patients with cancers susceptible to NK cell lysis, including blood cancers, such as acute myeloid leukemia or multiple myeloma, and several solid tumors, e.g., brain tumor, Ewing sarcoma and rhabdomyosarcoma. Increased numbers of functional NK cells can also significantly enhance the efficacy of therapeutic antibodies used in treatment of several cancers, including lymphomas, colorectal cancer, lung cancer, and breast cancer, among others.

Accordingly, in one embodiment, the adoptive NK cell therapy is administered to a subject having cancer, where the expanded NK cell preparation is administered to cause cancer cell death. Exemplary solid tumors suitable for treatment with the expanded NK cell preparations disclosed herein include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the expanded NK cell preparations provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, neuroblastoma, glioblastoma, and nasopharyngeal carcinoma and melanoma.

In some embodiments, the expanded NK cell preparation generated in accordance with the methods disclosed herein is administered to a subject having cancer in combination with a primary cancer treatment. In some embodiments, the expanded NK cell population is administered in combination with chemotherapy, surgery, or radiation. In some embodiments, the expanded NK cell preparation generated in accordance with the methods disclosed herein is administered to a subject having received an autologous stem cell transplant as a cancer treatment, e.g., in subjects having multiple myeloma.

In another embodiment, the adoptive NK cell therapy is administered to a subject having a viral infection, where the expanded NK cell preparation is administered to enhance anti-viral immunity and death of virus infected host cells. Viral infections suitable for treatment with the expanded NK cell preparations generated in accordance with the methods disclosed herein include any infection caused by or associated with a double-stranded DNA (dsDNA), single-stranded DNA (ssDNA), double-stranded genomic RNA (dsRNA), single-strand positive RNA, and single-strand negative RNA virus. In any embodiment, the viral infection is an acute infection, for example and without limitation, infection by influenza (e.g., H1N1, H5N1), parainfluenza, paramyxovirus, adenovirus, parvovirus, enterovirus, variola virus, rotavirus, flavivirus infections (e.g., dengue virus (DENV), West Nile virus, Japanese encephalitis virus, tick-borne encephalitis virus, yellow fever virus and Zika virus), hemorrhagic fever viruses (e.g., viruses in the families of Arenaviridae, Bunyaviridae, Filoviridae, Falviviridae, and Togaviridae), and coronavirus (e.g., SARS-CoV, SARS-CoV-2, MERS-CoV). In any embodiment, the viral infection is a chronic viral infection, such as hepatitis virus, Epstein-Barr virus (herpes virus), human immunodeficiency virus (HIV).

Methods of administering cell compositions, such as compositions comprising a therapeutically effective amount of the expanded NK cells described herein are known in the art and include procedures such as those exemplified in U.S. Pat. Publ. Nos. 20180353544 to Rezvani et al. and 20210230548 to Daher et al., which are hereby incorporate by reference in their entirety. The amount of activated NK cells used can vary between in vitro and in vivo uses, as well as with the amount and type of the target cells. The amount administered will also vary depending on the condition of the patient and should be determined by considering all appropriate factors by the practitioner.

### EXAMPLES

The following examples are provided to illustrate embodiments of the present disclosure but are by no means intended to limit its scope.

### Example 1: Feeder-Free Culture System Achieves Significant Expansion of NK Cell Populations

A starting population of NK cells originated from CD34⁺ cord blood was obtained for establishment of the feeder-free expansion culture. This starting preparation of NK cells was primarily composed of immature NK cells that were CD56^{+/-}/CD16⁻/CD45⁺/CD34⁻.

Several cultures, each containing approximately 5×10⁵ cells in 10 ml of NK-MACS medium (Miltenyi) containing 5% FBS, were seeded on day zero (seeding day). To each culture, a certain combination of expansion factors was added to investigate their ability to induce NK cell expansion. These combinations included anti-NKp30 monoclonal antibody conjugated beads (NKp30)/anti-2B4 monoclonal antibody conjugated beads (2B4) (0.25 ug/ml), NKp30/2B4/CD155-Fc conjugated beads (CD155) (NKp30/2B4 at 0.25 ug/ml and CD155 at 3 ug/ml); NKp30/2B4/ICAM2-Fc conjugated beads (ICAM2) (NKp30/2B4 at 0.25 ug/ml and ICAM2 at 3 ug/ml), NKp30/2B4/4-1BBL-Fc conjugated beads (4-1BBL) (NKp30/2B4 at 0.25 ug/ml and 4-1BBL at 3 ug/ml); and NKp30/CD48-Fc conjugated beads (CD48) (NKp30 at 0.25 ug/ml and CD48 at 3 ug/ml). For comparison, seeded NK cultures were also grown in the presence of traditional feeder cells, i.e., K562 cells, an myelogenous leukemia cell line, or the K562 feeder cells in combination with 4-1BBL and IL21 cytokines. The cells were incubated in standard culture conditions, i.e., 37° C., 5% CO₂, and 80% humidity).

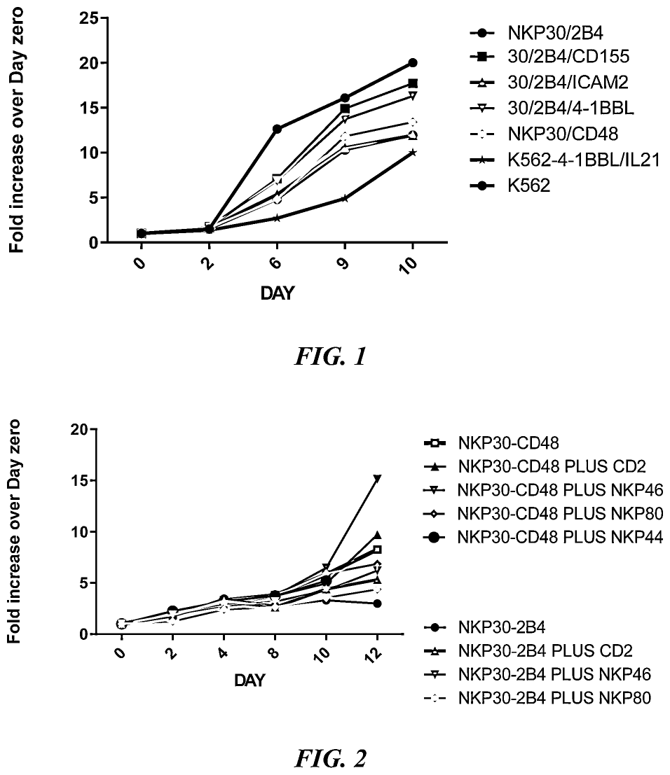

FIG. 1 shows the increase in NK cell expansion observed over 10 days in culture in the presence of the various expansion agents as described in the preceding paragraph. The expanded cell population was characterized as a mixture of immature and mature NK cells based on an expression profile of CD56+/CD3−/CD45+/CD16^{+/-}. As shown in the graph of FIG. 1, over this short culture period of 10 days, a >15-fold increase in the number of NK cells was observed in the cultures treated with the combination of NKp30 antibody/2B4 antibody/CD155 and the combination of NKp30 antibody/2B4 antibody/ICAM2. The fold-expansion in these cultures corresponded to the level of NK expansion observed feeder cultures.

In a second round of culture, cells cultured in the presence of NKp30/2B4 from the first round described in the preceding paragraph were chosen for a second round of expansion using the same or different combination of expansion factors. Without removing initial leftover beads, the cell number was adjusted to initial optimum number (2.5E5/ml), then fresh NKp30/2B4 beads (0.25 ug/ml) or CD48 beads (3 ug/ml) were added. All other anti-CD2, NKp46, NKp44 and NKp80 were added at 0.25 ug/ml concentration at individual conjugated beads.

FIG. 2 shows the increase in NK cell expansion observed over an additional 12 days of culture. As shown in this figure, treating starting cultures of NK cells with NKp30 with CD48, the ligand for 2B4, had a higher fold-expansion as compared to cultures treated with NKp30 and the 2B4 antibody. The inclusion of other factors, particularly the NKp46 monoclonal antibody further enhanced expansion.

In a second experiment, the effect of the solid support, i.e., bead conjugate, was tested in the feeder-free cultures. In this experiment, anti-NKp30 antibody was conjugated to anti-biotin beads and was used at 0.25 ug/ml in all conditions. To test the solid support, CD48 and CD155 were conjugated to pierce A/G beads (described as AG condition on bar graph) or agarose beads containing anti-human Fc peptide (described as Ligatrap condition on Bar graph). For non-solid support condition, CD48 and CD155 was incubated with anti-human Fc monoclonal antibody without any beads conjugation (described as Fc condition on bar graph).

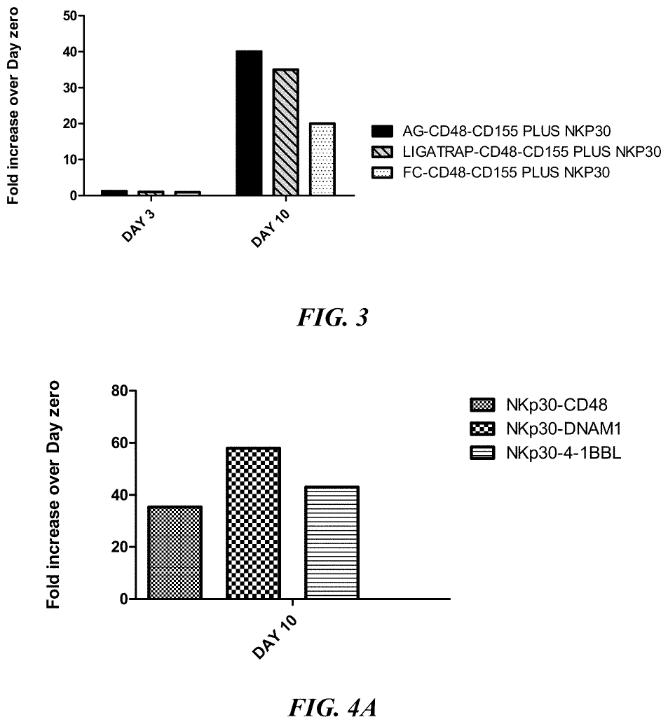

As shown in the graph of FIG. 3, NK cell expansion in cultures where the expansion factors were conjugated to protein A/G beads had the highest fold-expansion over 10 days as compared to the cultures containing factors conjugated to LigaTrap beads and anti-Fc beads.

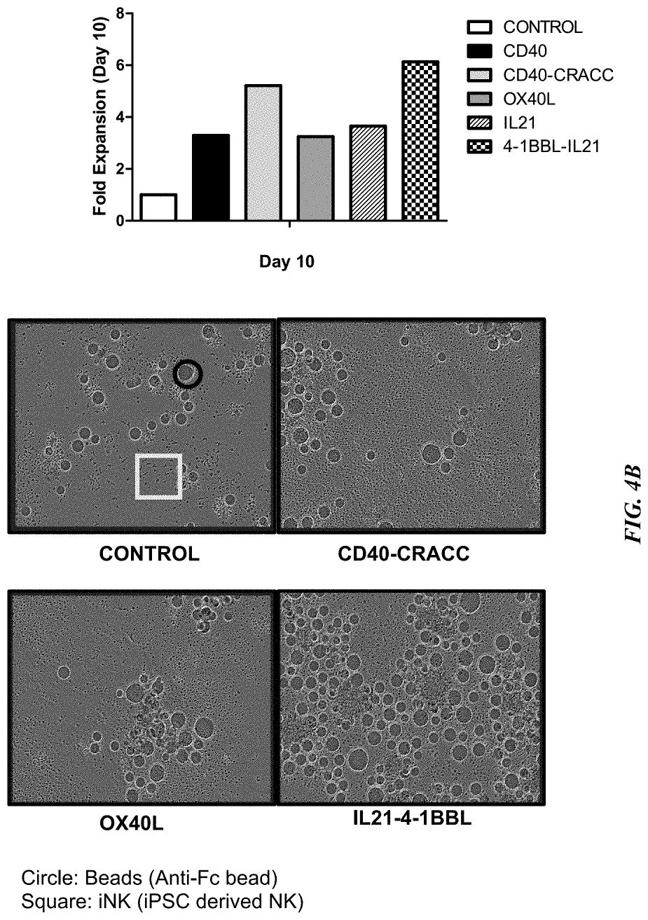

In a third experiment, the effect of DNAM1, CD48 and 4-1BBL in combination with NKp30 was tested on iNK expansion. As shown in FIG. 4, NK cell expansion in cultures treated with the combination of NKp30 and DNAM1 conjugated to anti biotin beads had the highest fold expansion over 10 days as compared to the cultures containing CD48 or 4-1BBL conjugated to LigaTrap beads.

### Example 2: Characterization of Feeder-Free Expanded NK Cell Populations

Flow cytometry analysis of feeder-free expanded NK cell populations was conducted to assess the heterogeneity of the population. Accordingly, three days of culture in the presence of NKp30 and an additional seven days of culture in the presence of anti DNAM1 biotinylated antibody conjugated to anti biotin beads, expression of general NK markers (CD56, NKP30, DNAM1, 2B4), immature NK markers (NKG2A, NKG2D, NKP46, NKP44) and mature NK markers (NKp80, CD16, KIR, CRACC) was assessed in CD56+CD3− sub gated populations of NK cells. In addition, the expression of exhaustion markers, i.e., PD1, LAG3, TIM3, TIGIT, was also expressed.

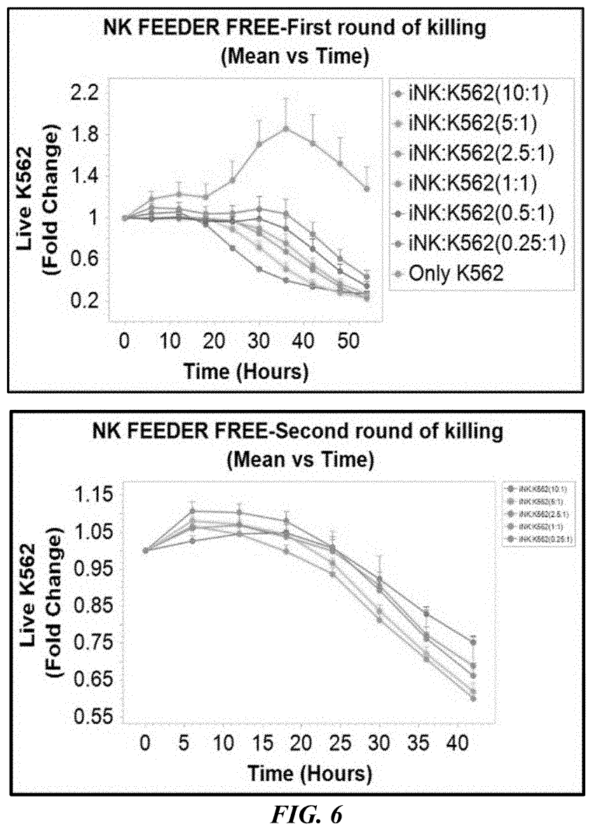

To evaluate the killing potency of feeder-free expanded NK populations toward cancer cells (K562), target cells were seeded in 96-well plates at optimal number (20K). This number was selected to avoid high cell density after addition of effector cells (NK cells). On the day of the assay, target cells settled for at least 2 h after seeding in triplicate in 96-well plates. Expanded NK cells were then added to the plate at various E:T ratios (NK: K562), and whole well images were recorded every 6 h for 50 h using Incucyte imaging system. The killing potency of the expanded NK cells toward cancer cells was assessed by comparing the number of fluorescent target cells at each time point to their number at time zero. Killing of cancer cells was readily detectable at the first 25-hour time point and increased over time as shown by the decrease in the percentage of live fluorescent K562 in the presence of the expanded INK population (FIG. 6). Killing was proportional to the ratio of NK cells and earliest killing occurred between E:T ratios of 10:1 and 2.5:1.

### Discussion of Examples 1 and 2

Human NK cells are a type of lymphocyte that characterized by the expression of CD56 or CD16 and the absence of CD3. NK cells express both activating receptors and inhibitory receptors. When their activating receptors, such as NKG2D, NKp30, 2B4, NKp44, NKp46, NKp80 and DNAM-1, are engaged, NK cells can directly kill target cells (e.g., cancer cells) which express the ligands for these activating receptors (such as B7H6 (NKp30 ligand), CD48(2B4 ligand) and CD155 (DNAM-1 ligand)). On the other hand, engagement of inhibitory receptors (NKG2A) on NK cells prevents the lysis of target cells.

Adoptive NK cell treatment is a promising therapy for cancer patients. To reach an effective therapeutic dose, NK cells need to be expanded in a large amount. However, previous studies have shown that NK cell expansion is limited to several divisions due to cell senescence. Current expansion methods to expand NK cells involve the use of high dose cytokines with activating ligands expressed on leukemia-derived feeder cell lines, such as K562 cells. The use of feeder cells introduces unknown variables into the NK cell production system, which is unfavorable to the safety and quality of NK cell product for clinical usage. The feeder-free expansion culture protocol described herein eliminates the need for feeder cells, thus simplifying the methodology yet providing comparable levels of cell expansion.

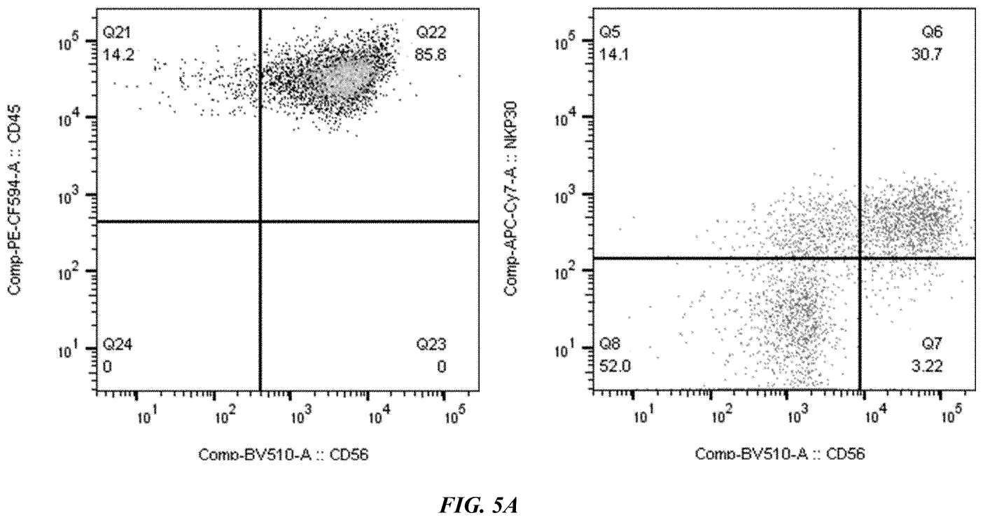

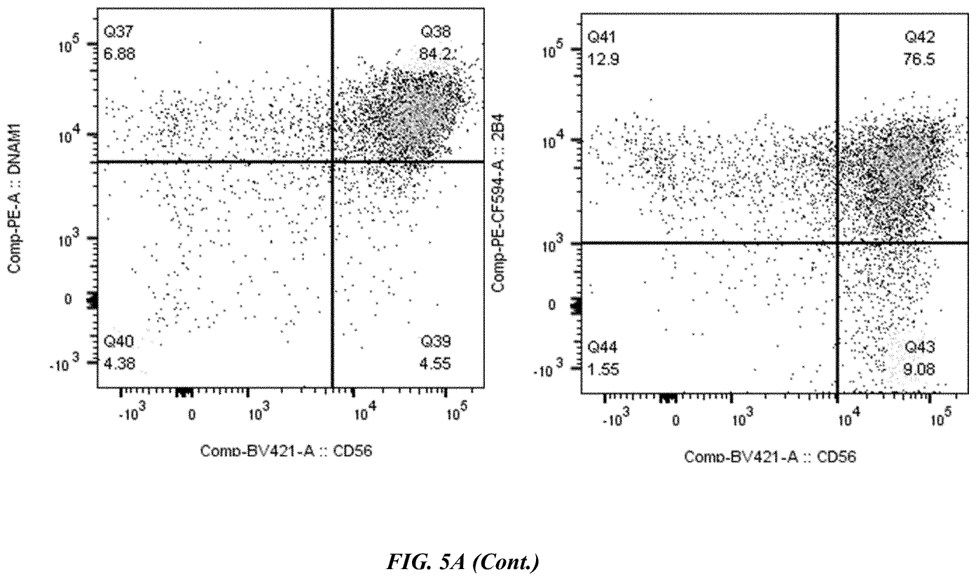

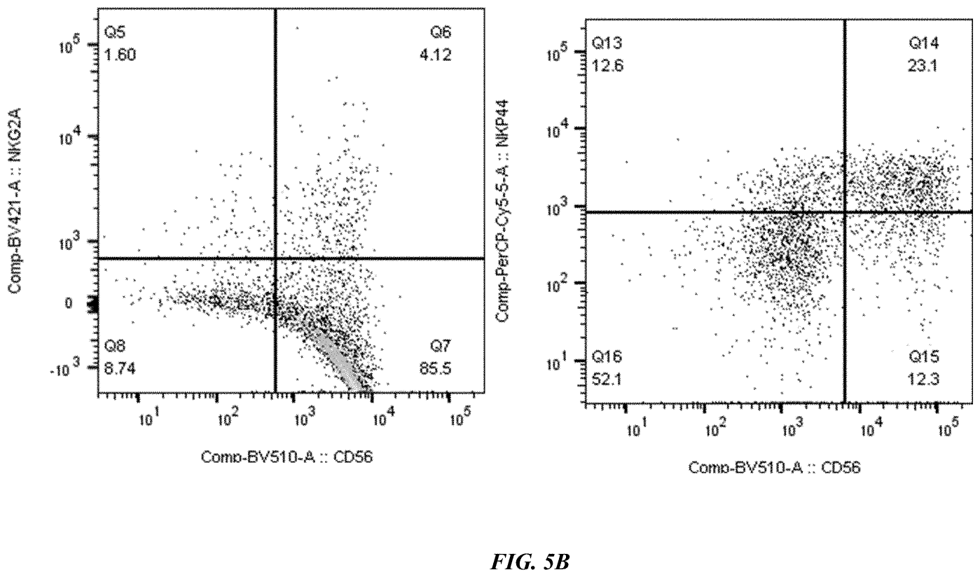

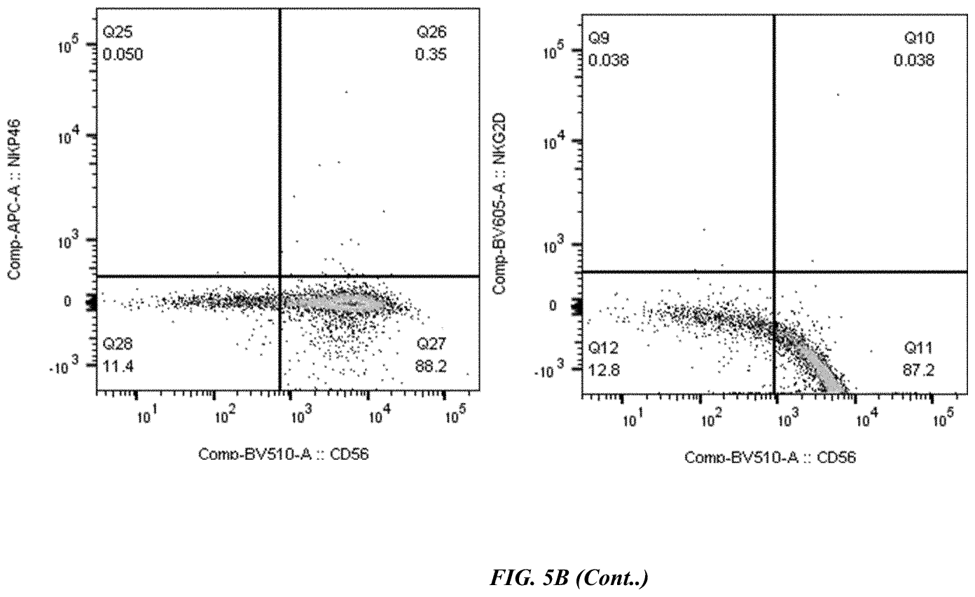

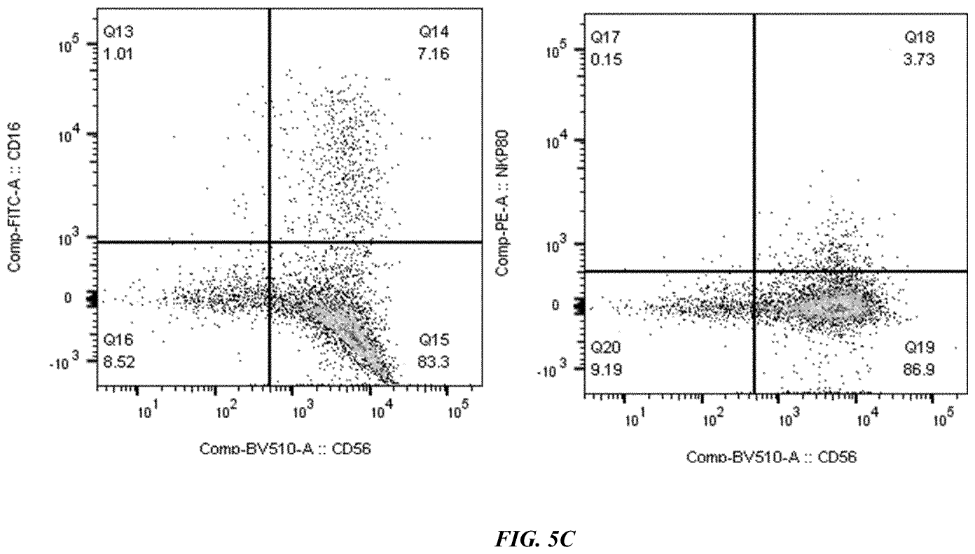

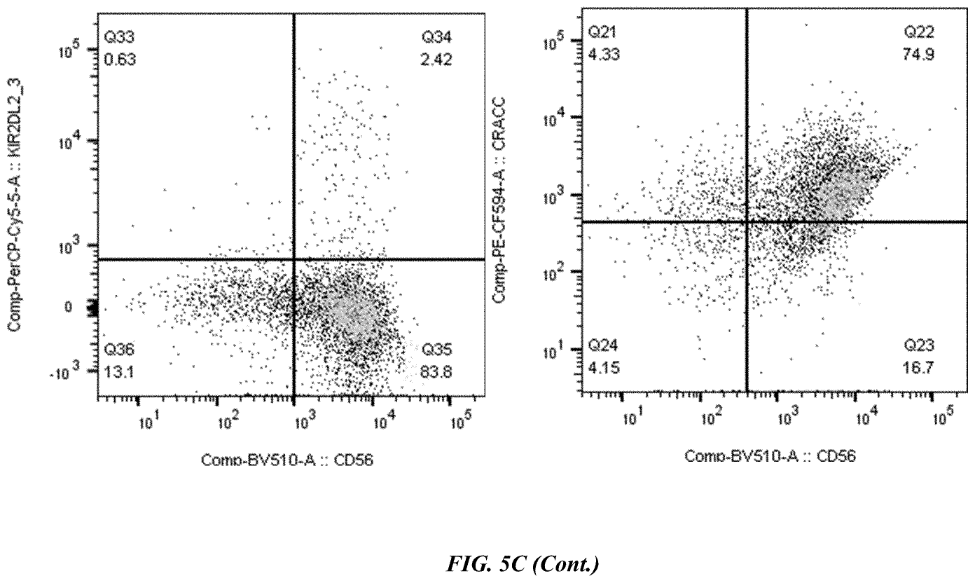

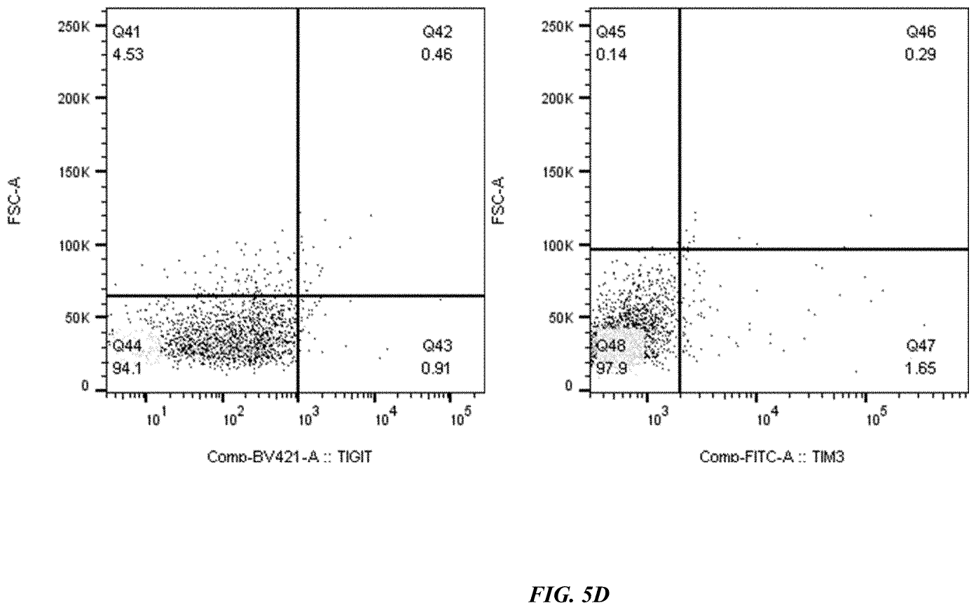

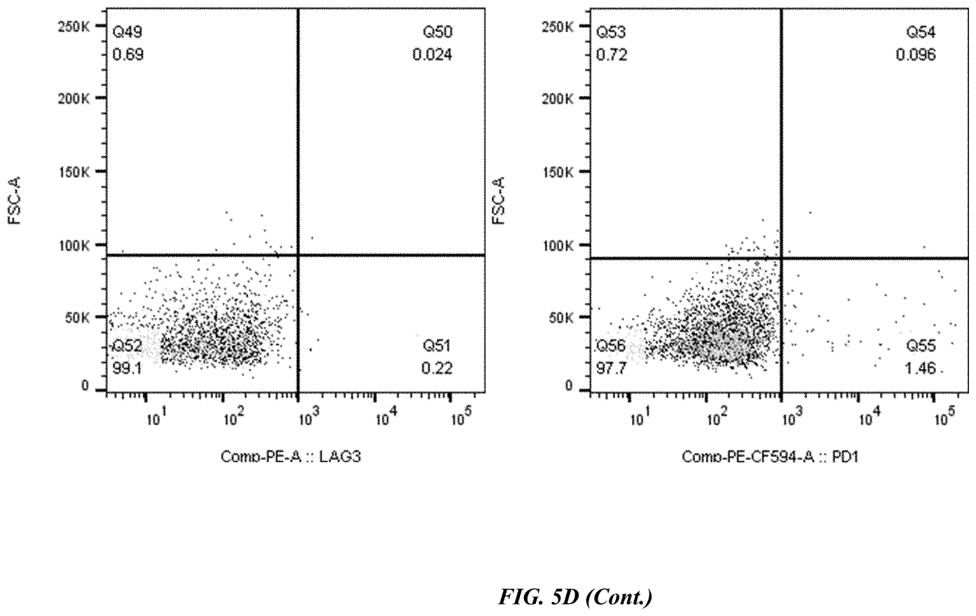

In addition, as shown by the flow cytometry analysis of expanded NK populations produced as described herein (FIG. 5A-C), the feeder free system produces a mixture of late stage immature NK cells as indicated by the expression of NKG2A, NKG2D, NKP46, NKP44 (FIG. 5B) and mature NK as indicated by the expression of NKp80, CD16, KIR, CRACC (FIG. 5C). On the other hand, as shown in the flow cytometry analysis of FIG. 5D, feeder-free expanded NK populations did not show any sign of exhaustion as indicated by a lack of expression of known NK exhaustion markers such as, PD1, LAG3, TIM3, and TIGIT. This lack of exhaustion and presence of both immature and mature status of expanded NK as shown in FIGS. 5A-D, should be advantageous from the perspective of persistence and expansion capacity upon transplantation into the patient. In contrast, NK cells expanded in the feeder cell systems produce a more uniform population of mature NK cells that likely have limited expansion capacity and/or are closer to exhaustion.

As shown in FIG. 6, the cytotoxicity of NK cells expanded in the feeder free system was robust and comparable to the published cytotoxicity of NK cells expanded in the feeder system at least against nonspecific target (K562). Finally, since the feeder cells are tumorigenic, employing a feeder free system eliminates the need to employ stringent purification steps following expansion, as needed in the feeder system cell cultures to remove the tumorigenic cells.

Although preferred embodiments have been depicted and described in detail herein, it will be apparent to those skilled in the relevant art that various modifications, additions, substitutions, and the like can be made without departing from the spirit of the disclosure and these are therefore considered to be within the scope of the disclosure as defined in the claims which follow.

1. A method of producing a therapeutic preparation of natural killer (NK) cells, said method comprising:
providing a starting preparation of NK cells;
treating said starting preparation with a natural killer cell p30-related protein (NKp30) modulating agent; and
culturing the treated preparation under conditions effective to expand the starting preparation of NK cells thereby producing an expanded NK cell preparation.

2. The method of claim 1, wherein said culturing does not involve culturing the starting preparation of NK cells in the presence of a feeder cell population of cells.

3. The method of claim 1, wherein said NKp30 modulating agent is a NKp30 antibody.

4. The method of claim 1, wherein said treating further comprises:
administering to said preparation, in conjunction with the NKp30 modulating agent, a 2B4 ligand, a DNAM-1 ligand, or a combination thereof.

5. The method of any of claim 1, wherein the NKp30 modulating agent, the 2B4 ligand, and/or the DNAM-1 ligand are coupled to a solid support.

6. The method of claim 5, wherein the solid support is a plurality of beads.

7. The method of claim 4, wherein the 2B4 ligand is a CD48 protein or polypeptide fragment thereof.

8. The method of claim 4, wherein the 2B4 ligand is a 2B4 antibody or antibody binding fragment thereof.

9. The method of claim 4, wherein the DNAM-1 ligand is a CD155 protein or polypeptide fragment thereof.

10. The method of claim 4, wherein the DNAM-1 ligand is a CD112 protein or polypeptide fragment thereof.

11. The method of claim 1, wherein said treating comprises:
administering a combination of the NKp30 modulating agent, the 2B4 ligand, and the DNAM-1 ligand to the starting preparation of NK cells.

12. The method of claim 11, wherein the combination comprises an NKp30 antibody, a CD48 protein or polypeptide, and a CD155 protein or polypeptide.

13. The method of claim 1, wherein said treating is repeated periodically during said culturing.

14. The method of claim 1, wherein said treating and said culturing are effective to produce an expanded NK cell preparation comprising at least 20-fold more NK cells than the number of NK cells in the starting preparation.

15. The method of claim 1, wherein said treating and said culturing are effective to produce an expanded NK cell preparation comprising at least 40-fold more NK cells than the number of NK cells in the starting preparation.

16. The method of claim 1, wherein said treating and said culturing are effective to produce an expanded NK cell preparation comprising than 40-fold more NK cells than the number of NK cells in the starting preparation.

17. The method of claim 1, wherein the expanded NK cell preparation is a human expanded NK cell preparation.

18. The method of claim 1, wherein the expanded NK cell preparation comprises a heterogeneous mixture of late stage immature NK cells and mature NK cells.

19. The method of claim 1, wherein the expanded NK cell preparation comprises CD56⁺/CD3⁻/CD45⁺/CD16^{+/-} NK cells.

20. The method of claim 1, wherein the expanded NK cell preparation comprises cells expressing NKG2-C type II integral membrane protein but not expressing NKG2-A/NKG2-B type II integral membrane protein.

21. The method of claim 1, wherein cells of the starting preparation of NK cells are genetically modified, thereby producing an expanded NK cell preparation comprising genetically modified NK cells.

22. The method of claim 21, wherein the genetically modified NK cells express a chimeric antigen receptor (CAR).

23. The method of claim 1, wherein the NK cells of the starting preparation are CD56^{+/-}/CD16⁻/CD45⁺/CD34⁻ iNK cells.

24. The method of claim 23, wherein the starting preparation of NK cells are derived from CD34⁺ induced pluripotent stem cell (iPSCs) or CD34⁺ cord blood cells.

25. The method of claim 1, wherein said method further comprises:
administering one or more stimulatory cytokines to the starting preparation during said treating or said culturing.

26. The method of claim 25, wherein the one or more stimulatory cytokines is selected from IL-2, IL-12, IL-15, IL18, and IL21.

27. The method of claim 1 further comprising:
treating, after said culturing, the expanded preparation of NK cells with a DNAM-1 ligand in combination with one or more of a 2B4 ligand, 4-1BB ligand, or OX40L

28. The method of claim 1, wherein the expanded NK cell preparation comprises about 2×10⁹ to about 1×10¹¹ of NK cells.

29. A therapeutic preparation of NK cells produced according to the method of claim 1.

30. A pharmaceutical composition comprising:
the therapeutic preparation of NK cells of claim 29, and
a pharmaceutically acceptable carrier.

31. A method of treating a subject in need of adoptive NK cell therapy, said method comprising:
administering, to said subject, the pharmaceutical composition of claim 30 in an amount effective to treat the subject in need of adoptive NK cell therapy.

32. The method of claim 31, wherein said subject has cancer and said administering causes cancer cell death.

33. The method of claim 31 wherein said subject has a viral infection and said administering causes virus-infected cell death.
